(19) United States
(12) Patent Application Publication
(10) Pub. No.: US 2025/0092458 A1
(43) Pub. Date: Mar. 20, 2025

(54) Scaffolded Chromophores for Nucleic Acid Detection and Methods and Uses Thereof

(71) Applicant: Becton, Dickinson and Company, Franklin Lakes, NJ (US)

(72) Inventors: Glenn P. Bartholomew, San Marcos, CA (US); Jody Martin, Ashland, OR (US)

(21) Appl. No.: 18/821,342

(22) Filed: Aug. 30, 2024

Related U.S. Application Data
(60) Provisional application No. 63/539,067, filed on Sep. 18, 2023.

(51) Int. Cl. C12Q 1/6876; B01L 7/00

(52) U.S. Cl. CPC C12Q 1/6876; B01L 7/52; B01L 2200/0647; B01L 2300/025; B01L 2300/1805; B01L 2300/1833

(57) ABSTRACT

Scaffolded chromophores for nucleic acid detection and systems, methods, and uses thereof are provided. Certain embodiments are directed to nucleic acid probes that include a nucleic acid that is complementary to a target sequence. The probe further includes a dye structure linked to a first end of the nucleic acid and includes a non-conjugated polymeric backbone with one or more donor fluorophores linked to the polymeric backbone and one or more acceptor fluorophores linked to the polymeric backbone, where the donor and acceptor fluorophores are in energy transfer relationship. Such probes can further include a quencher attached to a second end of the nucleic acid, where the quencher and one or more acceptor fluorophores are in an energy transfer relationship. Additional embodiments include a second nucleic acid probe including a second nucleic acid that is complementary to a different target sequence.

### CROSS-REFERENCE TO RELATED APPLICATIONS

This application claims priority to the filing date of U.S. Provisional Patent Application Ser. No. 63/539,067 filed on Sep. 18, 2023; the disclosure of which application is incorporated herein by reference.

### INTRODUCTION

Nucleic acid detection has many uses in science and medicine, such as diagnostics and/or environmental monitoring. However, detection relies on fluorescently labeled probes, which may be subject to certain limitations, including a lack sensitivity, faintness, or an inability for multiplexing. Because of these limitations, there is a need for probes capable of being tuned for increased fluorescence emission and/or multiplexing.

### SUMMARY

Scaffolded chromophores for nucleic acid detection and systems, methods, and uses thereof are provided. Certain embodiments are directed to nucleic acid probes that include a nucleic acid that is complementary to a target sequence. The probe further includes a dye structure linked to a first end of the nucleic acid and includes a non-conjugated polymeric backbone with one or more donor fluorophores linked to the polymeric backbone and one or more acceptor fluorophores linked to the polymeric backbone, where the donor and acceptor fluorophores are in energy transfer relationship. Such probes can further include a quencher attached to a second end of the nucleic acid, where the quencher and one or more acceptor fluorophores are in energy transfer relationship. Additional embodiments include a second nucleic acid probe including a second nucleic acid that is complementary to a different target sequence.

Probes of certain embodiments can include DNA, RNA, and/or locked nucleic acid. Individual bases can include canonical bases, modified bases, base analogs, minor groove binders, water solubilizing groups, and/or additional quenchers. The non-conjugated polymeric backbone can be comprised of one or more of a peptide, a carbohydrate, a hydrocarbon, a polyethylene glycol polymer, a lipid, a peptoid, and a polynucleotide.

Additional embodiments include methods, systems, kits, and hybridization products that include nucleic acid probes as described above. Such methods can be used to detect a target nucleic acid and include producing a reaction mixture that includes a sample and a nucleic acid probe and monitoring the reaction mixture for a signal emitted from the dye structure to assay the sample for the presence of the target nucleic acid. Such methods can include performing a nucleic acid amplification reaction, such as PCR and/or an isothermal amplification. Kits can include a probe as described above as well as one or more reagents to perform a reaction, such as a nucleic acid amplification reaction. Additional embodiments are directed to products that include a probe as described above hybridized to a target nucleic acid.

Systems include a sample chamber holding a sample, an illumination source directed to impinge on the sample, and a detector such that light emissions from the sample impinge on the detector. Additional systems can include a heating element, such as a heat block and/or water bath. Additional systems can include a user interface to allow selection of a temperature, an illumination wavelength, a detection wavelength, and temperature cycling program.

### BRIEF DESCRIPTION OF THE DRAWINGS

The invention may be best understood from the following detailed description when read in conjunction with the accompanying drawings. Included in the drawings are the following figures:

FIG. 1 provides a schematic nucleic acid probe having a scaffolded chromophore dye structure and a quencher in accordance with various embodiments of the invention.

FIG. 2 provides a non-limiting example of a nucleic acid probe having a scaffolded chromophore dye structure and a quencher in accordance with various embodiments of the invention.

### DEFINITIONS

As used herein, the terms “chemoselective functional group” and “chemoselective tag” are used interchangeably and refer to a functional group that can selectively react with another compatible functional group to form a covalent bond, in some cases, after optional activation of one of the functional groups. Chemoselective functional groups of interest include, but are not limited to, thiols and maleimide or iodoacetamide, amines and carboxylic acids or active esters thereof, as well as groups that can react with one another via Click chemistry, e.g., azide and alkyne groups (e.g., cyclooctyne groups), tetrazine, transcyclooctene, dienes and dienophiles, and azide, sulfur (VI) fluoride exchange chemistry (SuFEX), sulfonyl fluoride, as well as hydroxyl, hydrazido, hydrazino, aldehyde, ketone, azido, alkyne, phosphine, epoxide, and the like.

As used herein, the term “sample” relates to a material or mixture of materials, in some cases in liquid form, containing one or more analytes of interest—e.g., nucleic acids. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g., body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi. The term “sample” may further refer to an “environmental sample.” As used herein, the term “an environmental sample” refers to a swab taken from a non-biological location (e.g., a high-touch location, including, but not limited to a doorknob, toilet seat, faucets, water fountains, etc.). An “environmental sample” can also refer to a liquid (e.g., water, beverage, fuel, petroleum isolate), a gas (e.g., air, medical oxygen, industrial oxygen, industrial nitrogen), soil, filter, swab, and/or any other non-biological material. Such environmental samples may contain biological materials, including cells and/or nucleic acids.

The term “nucleic acid” refers to a polymeric form of nucleotides of any length, including nucleic acids that range from 2-100 nucleotides in length and nucleic acids that are greater than 50 nucleotides in length. The terms “nucleotide” refers to a sugar, a base, and a phosphate group. The terms “nucleobase” and “base” are used interchangeably herein. The term “nucleic acid” includes polymers of canonical (adenine (A), cytosine (C), guanine (G), thymine (T), uracil (U)) and non-canonical bases, chemically or biochemically modified or derivatized nucleotides, and nucleotides having modified sugar-phosphate backbones in which the conventional backbone has been replaced with non-naturally occurring or synthetic backbones. Conventional backbones are generally considered to be a ribose-phosphate backbone (as used in ribonucleic acid (RNA)) and a deoxyribose-phosphate backbone (as used in deoxyribonucleic acid (DNA)). Non-naturally occurring, synthetic, or otherwise non-conventional backbones, including replacing a ribose or deoxyribose with another sugar (e.g., threose), a peptide, or other moiety. Examples of non-naturally occurring, synthetic, or otherwise non-conventional backbones include xeno nucleic acid (XNA), peptide nucleic acid (PNA), morpholino nucleic acid, locked nucleic acid (LNA), glycol nucleic acid (GNA), 1,5-anhydrohexitol nucleic acid (HNA), Cyclohexene nucleic acid (CeNA), Fluoro Arabino nucleic acid (FANA), and threose nucleic acid (TNA). Certain nucleic acids may contain one or more nucleotides with a non-conventional backbone amongst conventional backbones—for example, 1 or more nucleotides may be LNA nucleotides, while the remaining nucleotides are DNA nucleotides. A nucleic acid may be of any convenient length, e.g., 2 or more nucleotides, such as 4 or more nucleotides, 10 or more nucleotides, 20 or more nucleotides, 50 or more nucleotides, 100 or more nucleotides, 300 or more nucleotides, such as up to 500 or 1000 or more nucleotides.

As used herein the term “isolated,” refers to a molety of interest that is at least 60% free, at least 75% free, at least 90% free, at least 95% free, at least 98% free, and even at least 99% free from other components with which the moiety is associated with prior to purification.

A “plurality” contains at least 2 members. In certain cases, a plurality may have 5 or more, such as 6 or more, 7 or more, 8 or more, 9 or more, 10 or more, 20 or more, 30 or more, 40 or more, 50 or more, 60 or more, 70 or more, 80 or more, 90 or more, 100 or more, 300 or more, 1000 or more, 3000 or more, 10,000 or more, 100,000 or more members.

Numeric ranges are inclusive of the numbers defining the range.

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced.

The specific binding member can be a nucleic acid. In nucleic acids, a particular nucleotide can pair with one or more other nucleotides, for example canonical pairing (adenine-thymine, cytosine-guanine) or ambiguous pairing (e.g., inosine-cytosine, inosine-adenine, etc.). A nucleic acid typically pairs with its reverse complement sequence, but some specific binding pairs may allow for ambiguity or mispairing to occur. Thus, the members of the pair have the property of binding specifically to each other. Such affinity or binding can generally be measured by its melting temperature (Tm), where a higher melting temperature is indicative of a stronger binding or higher affinity between the pair of nucleic acids. “Affinity” refers to the strength of binding, increased binding affinity being correlated with a higher melting temperature.

As used herein, the term “hybridization conditions” means conditions in which a primer, or other polynucleotide, specifically hybridizes to a region of a target nucleic acid with which the primer or other polynucleotide shares some complementarity. Whether a primer specifically hybridizes to a target nucleic acid is determined by such factors as the degree of complementarity between the polymer and the target nucleic acid and the temperature at which the hybridization occurs, which may be informed by the melting temperature (TM) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6 (log 10 [Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a region of the product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

As used herein, the term “excitation maximum” and “absorption maximum” refer to a peak absorption wavelength associated with a moiety. If such moieties are fluorescent (i.e., also emit a photon or other radiation), “excitation maximum” and “absorption maximum” may be used interchangeably. As used herein, the term “emission maximum” refers to a peak emission wavelength associated with a fluorescent moiety (e.g., fluorophore), where the highest intensity radiation (e.g., light) is emitted.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining,” “measuring,” and “assessing,” and “assaying” are used interchangeably and include both quantitative and qualitative determinations.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

As used herein, the terms “water solubilizing group”, “water soluble group” and WSG are used interchangeably and refer to a group or substituent that is well solvated in aqueous environments e.g., under physiological conditions, and which imparts improved water solubility upon the molecule to which it is attached. A WSG can increase the solubility of a probe, dye structure, or other component thereof, e.g., donor or acceptor fluorophore, in a predominantly aqueous solution, as compared to a control dye structure or component thereof which lacks the WSG. The water solubilizing groups may be any convenient hydrophilic group that is well solvated in aqueous environments.

A variety of water soluble polymer groups can be adapted for use in the WSG of the subject dyes. Any convenient water solubilizing groups (WSG's) may be included in the dyes described herein to provide for increased water-solubility. While the increase in solubility may vary, in some instances the increase (as compared to the compound without the WSG(s)) is 2 fold or more, e.g., 5 fold, 10 fold, 25 fold, 50 fold, 100 fold or more. In some cases, the hydrophilic water solubilizing group is charged, e.g., positively or negatively charged. In certain cases, the hydrophilic water solubilizing group is a neutral hydrophilic group. In some embodiments, the WSG is branched (e.g., as described herein). In certain instances, the WSG is linear. In some embodiments, the WSG is a hydrophilic polymer, e.g., a polyethylene glycol, a modified PEG, a peptide sequence, a peptoid, a carbohydrate, an oxazoline, a polyol, a dendron, a dendritic polyglycerol, a cellulose, a chitosan, or a derivative thereof. Water solubilizing groups of interest include, but are not limited to, carboxylate, phosphonate, phosphate, sulfonate, sulfate, sulfinate, sulfonium, ester, polyethylene glycols (PEG) and modified PEGs, hydroxyl, amine, amino acid, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, —COOM′, —SO₃M′, —PO₃M′, —NR₃⁺, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from O, S, and NR^{ZZ}, and R^{ZZ} and R^{YY} are independently selected from H and C₁₋₃ alkyl. In some cases, a WSG is (CH₂)ₓ(OCH₂CH₂)_{y}OCH₃ where each x is independently an integer from 0-20, each y is independently an integer from 0 to 50. In some cases, the water solubilizing group includes a non-ionic polymer (e.g., a PEG polymer) substituted at the terminal with an ionic group (e.g., a sulfonate).

In some embodiments of the formulae, the pendant group of interest includes a substituent selected from (CH₂)ₓ(OCH₂CH₂)_{y}OCH₃ where each x is independently an integer from 0-20, each y is independently an integer from 0 to 50; and a benzyl optionally substituted with one or more halogen, hydroxyl, C₁-C₁₂ alkoxy, or (OCH₂CH₂)_{z}OCH₃ where each z is independently an integer from 0 to 50. In some instances, the substituent is (CH₂)₃ (OCH₂CH₂)₁₁OCH₃. In some embodiments, one or more of the substituents is a benzyl substituted with at least one WSG groups (e.g., one or two WSG groups) selected from (CH₂)ₓ(OCH₂CH₂)_{y}OCH₃ where each x is independently an integer from 0-20 and each y is independently an integer from 0 to 50.

Multiple WSGs may be included at a single location in the subject dyes via a branching linker. In certain embodiments, the branching linker is an aralkyl substituent, further di-substituted with water solubilizing groups. As such, in some cases, the branching linker group is a substituent of the dye that connects the dye to two or more water solubilizing groups. In certain embodiments, the branching linker is an amino acid, e.g., a lysine amino acid that is connected to three groups via the amino and carboxylic acid groups. In some cases, the incorporation of multiple WSGs via branching linkers imparts a desirable solubility on the dye. In some instances, the WSG is a non-ionic sidechain group capable of imparting solubility in water in excess of 50 mg/mL. In some instances, the WSG is a non-ionic sidechain group capable of imparting solubility in water in excess of 100 mg/mL. In some embodiments, the dye includes substituent(s) selected from the group consisting of, an alkyl, an aralkyl and a heterocyclic group, each group further substituted with a include water solubilizing groups hydrophilic polymer group, such as a polyethylglycol (PEG) (e.g., a PEG group of 6-24 units).

Water soluble polymers of interest that can be utilized in the WSG include polyethylene glycol (PEG) groups or modified PEG groups. Water-soluble polymers of interest include, but are not limited to, polyalkylene oxide based polymers, such as polyethylene glycol “PEG” (See. e.g., “Poly(ethylene glycol) Chemistry: Biotechnical and Biomedical Applications”, J. M. Harris, Ed., Plenum Press, New York, N.Y. (1992); and “Poly(ethylene glycol) Chemistry and Biological Applications”, J. M. Harris and S. Zalipsky, Eds., ACS (1997); and International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966).

Examples of water soluble polymers of interest include, but are not limited to, those containing a polyalkylene oxide, polyamide alkylene oxide, or derivatives thereof, including polyalkylene oxide and polyamide alkylene oxide comprising an ethylene oxide repeat unit of the formula —(CH₂—CH₂—O)—. Further examples of polymers of interest include a polyamide having a molecular weight greater than 1,000 Daltons of the formula —[C(O)—X—C(O)—NH—Y—NH]n- or —[NH—Y—NH—C(O)—X—C(O)]ₙ—, where X and Y are divalent radicals that may be the same or different and may be branched or linear, and n is a discrete integer from 2-100, such as from 2 to 50, and where either or both of X and Y comprises a biocompatible, substantially non-antigenic water-soluble repeat unit that may be linear or branched. Further examples of water-soluble repeat units comprise an ethylene oxide of the formula —(CH₂—CH₂—O)— or —(O—CH₂—CH₂)—. The number of such water-soluble repeat units can vary significantly, with the number of such units being from 2 to 500, 2 to 400, 2 to 300, 2 to 200, 2 to 100, 6-100, for example from 2 to 50 or 6 to 50. An example of an embodiment is one in which one or both of X and Y is selected from: —((CH₂)ₙ₁—(CH₂—CH₂—O)ₙ₂—(CH₂)— or —((CH₂)ₙ₁—(O—CH₂—CH₂)ₙ₂—(CH₂)ₙ₋₁—), where n1 is 1 to 6, 1 to 5, 1 to 4, or 1 to 3, and where n2 is 2 to 50, 2 to 25, 2 to 15, 2 to 10, 2 to 8, or 2 to 5. A further example of an embodiment is one in which X is —(CH₂—CH₂)—, and where Y is —(CH₂—(CH₂—CH₂—O)₃—CH₂—CH₂—CH₂)— or —(CH₂—CH₂—CH₂—(O—CH₂—CH₂)₃—CH₂)—.

The term modified polymer, such as a modified PEG, refers to water soluble polymers that have been modified or derivatized at either or both terminals, e.g., to include a terminal substituent (e.g., a terminal alkyl, substituted alkyl, alkoxy or substituted alkoxy, etc.) and/or a terminal linking functional group (e.g., an amino or carboxylic acid group suitable for attachment via amide bond formation) suitable for attached of the polymer to a molecule of interest (e.g., to a light harvesting chromophore via a branching group). The subject water soluble polymers can be adapted to include any convenient linking groups. It is understood that in some cases, the water soluble polymer can include some dispersity with respect to polymer length, depending on the method of preparation and/or purification of the polymeric starting materials. In some instances, the water soluble polymers are monodisperse.

The water soluble polymer can include one or more spacers or linkers. Examples of spacers or linkers include linear or branched moieties comprising one or more repeat units employed in a water-soluble polymer, diamino and or diacid units, natural or unnatural amino acids or derivatives thereof, as well as aliphatic moieties, including alkyl, aryl, heteroalkyl, heteroaryl, alkoxy, and the like, which can contain, for example, up to 18 carbon atoms or even an additional polymer chain.

The water soluble polymer moiety, or one or more of the spacers or linkers of the polymer moiety when present, may include polymer chains or units that are biostable or biodegradable. For example, polymers with repeat linkages have varying degrees of stability under physiological conditions depending on bond lability. Polymers with such bonds can be categorized by their relative rates of hydrolysis under physiological conditions based on known hydrolysis rates of low molecular weight analogs, e.g., from less stable to more stable, e.g., polyurethanes (—NH—C(O)—O—)>polyorthoesters (—O—C((OR)(R′))—O—)>polyamides (—C(O)—NH—). Similarly, the linkage systems attaching a water-soluble polymer to a target molecule may be biostable or biodegradable, e.g., from less stable to more stable: carbonate (—O—C(O)—O—)>ester (—C(O)—O—)>urethane (—NH—C(O)—O—)>orthoester (—O—C((OR)(R′))—O—)>amide (—C(O)—NH—). In general, it may be desirable to avoid use of a sulfated polysaccharide, depending on the lability of the sulfate group. In addition, it may be less desirable to use polycarbonates and polyesters. These bonds are provided by way of example and are not intended to limit the types of bonds employable in the polymer chains or linkage systems of the water-soluble polymers useful in the WSGs disclosed herein.

The water soluble group (WSG) can be capable of imparting solubility in water in excess of 10 mg/mL to the subject probe or dye structure, such as in excess of 20 mg/mL, in excess of 30 mg/mL, in excess of 40 mg/mL, in excess of 50 mg/mL, in excess of 60 mg/mL, in excess of 70 mg/mL, in excess of 80 mg/mL, in excess of 90 mg/ml or in excess of 100 mg/mL. In certain cases, the branched non-ionic water soluble group (WSG) is capable of imparting solubility in water (e.g., an aqueous buffer) of 20 mg/mL or more to the subject probe or dye structure, such as 30 mg/ml or more, 40 mg/ml or more, 50 mg/mL or more, 60 mg/mL or more, 70 mg/mL or more, 80 mg/ml or more, 90 mg/mL or more, 100 mg/mL or more, or even more. It is understood that water-soluble dipyrromethene-based dye may, under certain conditions, form discrete water solvated nanoparticles in aqueous systems. In certain cases, the water solvated nanoparticles are resistant to aggregation and find use in a variety of biological assays.

The terms “polyethylene oxide”, “PEO”, “polyethylene glycol” and “PEG” are used interchangeably and refer to a polymeric group including a chain described by the formula —(CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use in the subject multichromophores include those PEGs described by S. Zalipsky in “Functionalized poly(ethylene glycol) for preparation of biologically relevant conjugates”, Bioconjugate Chemistry 1995, 6 (2), 150-165; and by Zhu et al in “Water-Soluble Conjugated Polymers for Imaging, Diagnosis, and Therapy”, Chem. Rev., 2012, 112 (8), pp 4687-4735.

The term “alkyl” by itself or as part of another substituent refers to a saturated branched or straight-chain monovalent hydrocarbon radical derived by the removal of one hydrogen atom from a single carbon atom of a parent alkane. Alkyl groups of interest include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group includes from 1 to 20 carbon atoms. In some embodiments, an alkyl group includes from 1 to 10 carbon atoms. In certain embodiments, a lower alkyl group includes from 1 to 6 carbon atoms, such as from 1 to 4 carbon atoms. This term includes, by way of example, linear and branched hydrocarbyl groups such as methyl (CH₃—), ethyl(CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃) (CH₃CH₂) CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkoxy” refers to the group-O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

“Alkenyl” refers to a monoradical, branched or linear, cyclic or non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Alkynyl” or “alkyne” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C═CH), and propargyl (—CH₂C═CH).

The term “substituted alkynyl” or “substituted alkyne” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Heterocyclyl” refers to a monoradical, cyclic group that contains a heteroatom (e.g., O, S, N) in as a ring atom and that is not aromatic (i.e., distinguishing heterocyclyl groups from heteroaryl groups). Exemplary heterocyclyl groups include piperidinyl, tetrahydrofuranyl, dihydrofuranyl, and thiocanyl.

“Amino” refers to the group —NH₂. The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen. “Aryl” by itself or as part of another substituent refers to a monovalent aromatic hydrocarbon radical derived by the removal of one hydrogen atom from a single carbon atom of an aromatic ring system. Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

“Substituted aryl”, unless otherwise constrained by the definition for the aryl substituent, refers to an aryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Heteroaryl” by itself or as part of another substituent, refers to a monovalent heteroaromatic radical derived by the removal of one hydrogen atom from a single atom of a heteroaromatic ring system. Heteroaryl groups of interest include, but are not limited to, groups derived from acridine, arsindole, carbazole, B-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, triazole, benzotriazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like. “Substituted heteroaryl”, unless otherwise constrained by the definition for the substituent, refers to an heteroaryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

The term “alkaryl” or “aralkyl” refers to the groups-alkylene-aryl and substituted alkylene-aryl where alkylene, substituted alkylene and aryl are defined herein. “Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C. (O)—, —C(O) NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like. “Substituted alkylene” refers to an alkylene group having from 1 to 3 hydrogens replaced with substituents as described for carbons in the definition of “substituted” below.

“Substituted” refers to a group in which one or more hydrogen atoms are independently replaced with the same or different substituent(s). Substituents of interest include, but are not limited to, alkylenedioxy (such as methylenedioxy), —M, —R⁶⁰, —O—, ═O, —OR⁶⁰, —SR⁶⁰, —S, ═S, —NR⁶⁰R⁶¹, ═NR⁶⁰, —CF₃, —CN, —OCN, —SCN, —NO, —NO₂, ═N₂, —N₃, —S(O)₂O, —S(O)₂OH, —S(O)₂R⁶⁰, —OS(O)₂₀, —OS(O)₂R⁶⁰, —P(O)(O)₂, —P(O)(OR⁶⁰) (O—), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —C(S) OR⁶⁰, —NR⁶²C(O) NR⁶⁰R⁶¹, —NR⁶²C(S) NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include —M, —R⁶⁰, ═O, —OR⁶⁰, —SR⁶⁰, —S, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂₀, —OS(O)₂R⁶⁰, —P(O)(O—)₂, —P(O)(OR⁶⁰)(O—), —OP(O)(OR⁶⁰)(OR⁶¹), —C(O)R⁶⁰, —C(S)R⁶⁰, —C(O)OR⁶⁰, —C(O)NR₆₀R⁶¹, —C(O)O″, —NR₆₂C(O)NR₆₀R⁶¹. In certain embodiments, substituents include —M, —R⁶⁰, ═O, —OR⁶⁰, —SR⁶⁰, —NR₆₀R⁶¹, —CF₃, —CN, —NO₂, —S(O)₂R⁶⁰, —P(O)(OR⁶⁰)(O—), —OP(O)(OR⁶⁰)(OR⁶¹), —C(O)R⁶⁰, —C(O)OR⁶⁰, —C(O)NR₆₀R⁶¹, —C(O) O″. In certain embodiments, substituents include —M, —R⁶⁰, ═O, —OR⁶⁰, —SR⁶⁰, —NR₆₀R⁶¹, —CF₃, —CN, —NO₂, —S(O)₂R⁶⁰, —OP(O)(OR⁶⁰)(OR⁶¹), —C(O) R⁶⁰, —C(O)OR⁶⁰, —C(O)O, where R⁶⁰, R⁶¹ and R⁶² are as defined above. For example, a substituted group may bear a methylenedioxy substituent or one, two, or three substituents selected from a halogen atom, a (1-4C) alkyl group and a (1-4C) alkoxy group. When the group being substituted is an aryl or heteroaryl group, the substituent(s) (e.g., as described herein) may be referred to as “aryl substituent(s)”. It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

“Acyl” refers to a group of formula —C(O) R wherein R is alkyl, alkenyl, or alkynyl. For example, the acetyl group has formula —C(O) CH₃. “Halo” and “halogen” refer to the chloro, bromo, fluoro, and iodo groups. “Carboxyl”, “carboxy”, and “carboxylate” refer to the —CO₂H group and salts thereof.

“Sulfonyl” refers to the group-SO₂R, wherein R is alkyl, alkenyl, alkynyl, aryl, heteroaryl, heterocyclyl, and substituted versions thereof. Exemplary sulfonyl groups include —SO₂CH₃ and —SO₂(C₆H₅).

Unless otherwise specified, reference to an atom is meant to include all isotopes of that atom. For example, reference to H is meant to include 1H, 2H (i.e., D) and 3H (i.e., T), and reference to C is meant to include 12C and all isotopes of carbon (such as 13C). In addition, any groups described include all stereoisomers of that group.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

### DETAILED DESCRIPTION

Scaffolded chromophores for nucleic acid detection and methods and uses thereof are provided. Scaffolded chromophores of embodiments of the invention include: a probe formed of a nucleic acid that is complementary to a target sequence; a dye structure linked to a first end of the nucleic acid that includes a non-conjugated polymeric backbone, one or more excitable donor fluorophores linked to the non-conjugated polymeric backbone, and one or more acceptor fluorophores linked to the non-conjugated polymeric backbone, where the donor and acceptor fluorophores are in energy transfer relationship; and a quencher linked to a second end of the nucleic acid, where the quencher and one or more acceptor fluorophores are in energy transfer relationship. Also provided are methods of making and using the scaffolded chromophores for nucleic acid detection, as well as kits that include the dyes and find use in embodiments of the methods.

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Still, certain terms are defined below for the sake of clarity and ease of reference. Further, although any methods and materials similar or equivalent to those described herein can also be used in the practice or testing of the present invention, representative illustrative methods and materials are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing certain embodiments of the invention, scaffolded chromophores for nucleic acid detection are reviewed first in greater detail, followed by a review of methods of using and making the dyes, as well as a review of kits that include the scaffolded chromophores for nucleic acid detection.

### Scaffolded Chromophores for Nucleic Acid Detection

As summarized above, the present disclosure provides scaffolded chromophores for use in nucleic acid detection. As used herein, “scaffolded chromophores” refers to chromophores, including fluorophores, arranged in an energy transfer relationship, such as FRET. Scaffolded chromophores include donor and acceptor chromophores arranged on a common structure, such as a backbone. Detection can be qualitative (e.g., presence/absence), quantitative (e.g., amount of target present), and/or semi-quantitative (e.g., relative amount of target). Scaffolded chromophores can be used as probes in nucleic acid amplification reactions, including Polymerase Chain Reaction (PCR) and its variations, such as quantitative PCR (qPCR), digital PCR (dPCR), and other relevant forms of PCR. Further methods of amplification include isothermal amplification reactions, such as Nucleic Acid Sequence-based Amplification (NASBA), Loop-mediated Isothermal Amplification (LAMP), Strand Displacement Amplification (SDA), Recombinase Polymerase Amplification (RPA), and Rolling Circle Amplification (RCA).

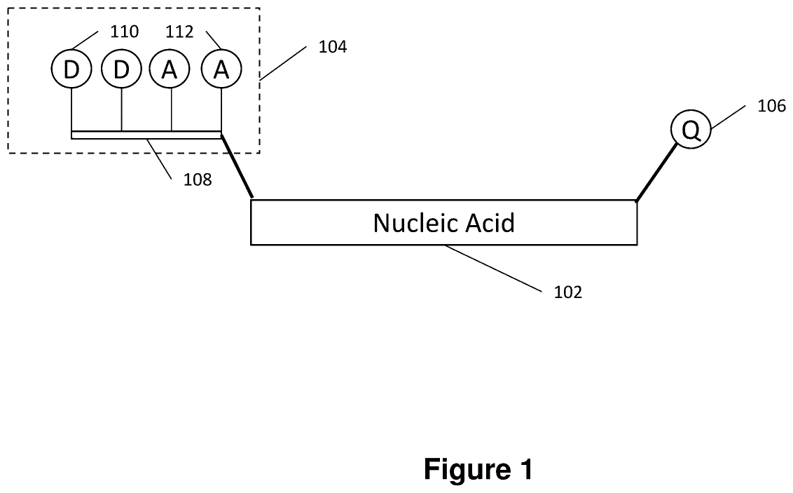

As further summarized above and illustrated in FIG. 1, certain embodiments are directed to a probe for identifying a target nucleic acid. To detect a target nucleic acid, certain embodiments comprise a nucleic acid 102 that is complementary to a target sequence. In certain embodiments, the nucleic acid is connected to a dye structure 104 and a quencher 106. Additional details of each component will be discussed further in the following text.

### Nucleic Acid

The nucleic acid 102 is comprised of a plurality of nucleotides forming a chain from a 5′-end to a 3′-end. The nucleic acid 102 may be of any length sufficient to hybridize to and/or be specific to a particular target sequence under particular conditions, such as reaction conditions for an amplification reaction. A temperature to allow hybridization to a target sequence can be measured by the melting temperature (or Tm) of a particular sequence. Tm is commonly measured and determinable using various available methods. In certain embodiments, the nucleic acid 102 may be of any convenient length, e.g., 2 or more nucleotides, such as 4 or more nucleotides, 10 or more nucleotides, 20 or more nucleotides, 50 or more nucleotides, 100 or more nucleotides, 300 or more nucleotides, such as up to 500 or 1000 or more nucleotides. Some embodiments include a nucleic acid having 10-50 nucleotides. Certain embodiments include a nucleic acid having 18-30 nucleotides.

The nucleic acid 102 and/or individual nucleotides can be formed of canonical bases (adenine (A), cytosine (C), guanine (G), thymine (T), uracil (U)) and non-canonical bases (also referred to as analogs), chemically or biochemically modified or derivatized nucleotides, and nucleotides having modified sugar-phosphate backbones in which the conventional backbone has been replaced with non-naturally occurring or synthetic backbones.

Various nucleotide modifications and analogs are known in the art, including 1-methyladenosine, 2-methylthio-N6-hydroxynorvalyl carbamoyladenosine, 2-methyladenosine, 2—O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl) adenosine, N6-hydroxynorvalylcarbamoyladenosine, 1,2-O-dimethyladenosine, N6,2-O-dimethyladenosine, 2-O-methyladenosine, N6,N6, O-2-trimethyladenosine, 2-methylthio-N6-(cis-hydroxyisopentenyl) adenosine, 2-methylthio-N6-methyladenosine, 2-methylthio-N6-isopentenyladenosine, 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine, N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N2-dimethylguanosine, 4-demethylwyosine, epoxyqueuosine, hydroxywybutosine, isowyosine, N2,7,2-O-trimethylguanosine, N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl) uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl) uridine methyl ester, dihydrouridine, 5-methyldihydrouridine, 5-methylaminomethyl-2-thiouridine, 5-(carboxyhydroxymethyl) uridine, 5-(isopentenylaminomethyl) uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl) pseudouridine, 1-methylpseudouridine, 3-methylpseudouridine, 2-O-methylpseudouridine, inosine, 1-methylinosine, 1,2-O-dimethylinosine and 2-O-methylinosine. Each of these may be components of nucleic acids of the present disclosure.

Conventional backbones are generally considered to be a ribose-phosphate backbone (as used in ribonucleic acid (RNA)) and a deoxyribose-phosphate backbone (as used in deoxyribonucleic acid (DNA)). Non-naturally occurring, synthetic, or otherwise non-conventional backbones, including replacing a ribose or deoxyribose with another sugar (e.g., threose), a peptide, or other moiety. Examples of non-naturally occurring, synthetic, or otherwise non-conventional backbones include xeno nucleic acid (XNA), peptide nucleic acid (PNA), morpholino nucleic acid, locked nucleic acid (LNA), glycol nucleic acid (GNA), 1,5-anhydrohexitol nucleic acid (HNA), Cyclohexene nucleic acid (CeNA), Fluoro Arabino nucleic acid (FANA), and threose nucleic acid (TNA). Certain nucleic acids may contain one or more nucleotides with a non-conventional backbone amongst conventional backbones—for example, 1 or more nucleotides may be LNA nucleotides, while the remaining nucleotides are DNA nucleotides.

### Dye Structure

Dye structure 104 can be constructed as scaffolded chromophores. In certain embodiments, dye structure 104 includes a polymeric backbone 108, one or more donor fluorophores 110 and one or more acceptor fluorophores 112. Donor 110 and acceptor 112 fluorophores are attached to the polymeric backbone 108. In certain embodiments, the polymeric backbone is non-conjugated. In certain embodiments, the dye structure 104 is attached to an end (e.g., 5′-end or 3′-end) of nucleic acid 102. In certain embodiments, the dye structure 104 is attached to a 5′-end of nucleic acid 102.

### Polymeric Backbone

As summarized above, dye structures of embodiments include a polymeric backbone 108. In some instances, the polymeric backbone 108 is made up of non-conjugated repeat units having any convenient configuration, such as a linear, branched or dendrimer configuration. The polymeric backbone 108 can be a linear polymer. The polymeric backbone 108 can be branched. In some instances, the dye structure includes a plurality of pendant donor chromophore groups (e.g., donor 110 and acceptor 112 fluorophores) each independently linked to a non-conjugated repeat unit of the polymeric backbone. The configuration of pendant groups can be installed during or after synthesis of the polymeric backbone. The incorporation of pendant groups can be with achieved with a random configuration, a block configuration, or in a sequence-specific manner via stepwise synthesis, depending on the particular method of synthesis utilized.

The term “unit” refers to a structural subunit of a polymer. The term unit is meant to include monomers, co-monomers, co-blocks, repeating units, and the like. A “repeating unit” or “repeat unit” is a subunit of a polymer that is defined by the minimum number of distinct structural features that are required for the unit to be considered monomeric, such that when the unit is repeated n times, the resulting structure describes the polymer or a block thereof. In some cases, the polymer may include two or more different repeating units, e.g., when the polymer is a multiblock polymer, a random arrangement of units or a defined sequence, each block may define a distinct repeating unit. It is understood that a variety of arrangements of repeating units or blocks are possible and that in the depicted formula of the polymer backbones described herein any convenient linear arrangements of various lengths can be included within the structure of the overall polymer. It is understood that the polymer may also be represented by a formula in terms of mol % values of each unit in the polymer and that such formula may represent a variety of arrangements of repeat unit, such as random or multiblock polymer or a defined sequence of residues. In some cases, a repeating unit of the polymer includes a single monomer group. In certain instances, a repeating unit of the polymer includes two or more monomer groups, i.e., co-monomer groups, such as two, three, four or more co-monomer groups. The term “co-monomer” or “co-monomer group” refers to a structural unit of a polymer that may itself be part of a repeating unit of the polymer.

The polymeric backbone 108 of the dye structure 104 may have any convenient length. In some cases, the particular number of monomeric repeating units or segments of the chromophore may fall within the range of 2 to 500,000, such as 2 to 100,000, 2 to 30,000, 2 to 10,000, 2 to 3,000 or 2 to 1,000 units or segments, or such as 5 to 100,000, 10 to 100,000, 100 to 100,000, 200 to 100,000, or 500 to 50,000 units or segments. In some instances, the particular number of monomeric repeating units or segments of the backbone may fall within the range of 2 to 1,000, such as 2 to 500, 2 to 100, 3 to 100, 4 to 100, 5 to 100, 6 to 100, 7 to 100, 8 to 100, 9 to 100 or 10 to 100 units or segments. In certain cases, the particular number of monomeric repeating units or segments of the backbone may fall within the range of 2 to 500, such as 2 to 400, 2 to 300, 2 to 200, or 2 to 100 units or segments. In certain cases, the particular number of monomeric repeating units or segments of the backbone may fall within the range of 2 to 100 repeating monomeric units, such as 2 to 90, 2 to 80, 2 to 70, 2 to 60, 2 to 50, 2 to 40, or 2 to 30 units or segments.

The polymeric backbone 108 may have a random configuration of non-conjugated repeat units. The polymeric backbone 108 may include a block or co-block configuration of non-conjugated repeat units. Alternatively, the polymeric backbone may include a particular defined sequence of non-conjugated repeat units, e.g., amino acid residues of a polypeptide sequence. These configurations can be characterized by polymeric segments of repeat units (e.g., as described herein), which segments can themselves be repeated throughout the modular scaffold.

By “non-conjugated” is meant that at least a portion of the repeat unit includes a saturated backbone group (e.g., a group having two or more consecutive single covalent bonds) which precludes pi conjugation or an extended delocalized electronic structure along the polymeric backbone from one repeat unit to the next. It is understood that even though one repeat unit may not be conjugated to an adjacent repeat unit, such a repeat unit may include one or more isolated unsaturated groups including an unsaturated bond (e.g., of an alkenylene group or an alkynylene group) and/or an aryl or heteroaryl group, which groups can be a part of the backbone. In some cases, each repeat unit of the polymeric backbone includes one sidechain including a linked pendant group or a chemo-selective tag for linking to a pendant group.

In certain embodiments of the dye structures 104, the polymeric backbone 108 is a linear polymer. In certain cases, the linear polymer is selected from a peptide, a peptoid, a hydrocarbon polymer, a PEG polymer, a carbohydrate, a lipid, and a polynucleotide. In certain cases, the linear polymer is a peptide. In certain cases, the linear polymer is a peptoid. In certain cases, the polymer is a hydrocarbon polymer. In certain other cases, the polymer is a PEG polymer. Further details regarding polymeric backbones that may be employed in embodiments of the invention are found in PCT application serial no. PCT/US2019/024662 published as WO2019/191482 and PCT application serial no. PCT/US2020/019510 published as WO2020/222894; the disclosures of which applications are herein incorporated by reference.

In certain instances, the dye structure includes a linear peptide backbone of from 2 to 100 amino acids, such as 2 to 90, 2 to 80, 2 to 70, 2 to 60, 2 to 50, 2 to 40 or 2 to 30 amino acids. In some cases, the linear peptide backbone includes 2 or more amino acids, such as 5 or more, 10 or more, 15 or more, 20 or more, 25 or more, 30 or more, up to a maximum of 100 amino acids. In certain cases, the dye structure includes a linear peptide backbone of from 5 to 30 amino acids, such as 5 to 25, 5 to 20, 5 to 15, or 5 to 10 amino acids.

Where desired, the polymeric backbones present in dye structures of embodiments of the invention may be substituted with one or more water solubilizing groups (WSG), e.g., as defined above.

### Donor and Acceptor Fluorophores

As provided above, dye structures of certain embodiments can include one or more donor fluorophores 110 and one or more acceptor fluorophores 112 each attached to the polymeric backbone 108. In certain embodiments, the donor 110 and acceptor 112 fluorophores are in energy transfer relationship. An energy transfer relationship is a situation in which emission from a donor fluorophore 110 is absorbed by an acceptor fluorophore 112. In certain instances, the emission maximum from a donor fluorophore 110 is at an excitation (or absorption) maximum of an acceptor fluorophore 112. The number of donor fluorophores 110 can be 1-20 fluorophores, including 1 to 20, 1 to 15, 1 to 10, 1 to 5, 2 to 20, 2 to 15, 2 to 10, 2 to 5, or any integer number of fluorophores between 1 and 20. Additionally, the number of acceptor fluorophores can be from 1-20 fluorophores, such as 1 to 20, 1 to 15, 1 to 10, 1 to 5, 1 to 4, 1 to 3, 1 to 2, or any integer number of fluorophores between 1 and 20.

By allowing various combinations or numbers of donor and acceptor fluorophores, certain embodiments allow for a tunable fluorescence mechanism for nucleic acid detection. Such tunability can come from varying the amount of donor and/or acceptor fluorophores within a dye structure. In some instances, light emission from an acceptor fluorophore may scale with the number of donor fluorophores, allowing for greater emission with the increase in number of donor fluorophores. Such a process can be used to increase sensitivity by a probe, such as to allow detection of minute or miniscule quantities of a target sequence. For example, to increase emission from an acceptor fluorophore, a probe may comprise more donor fluorophores may exceed the number of acceptor fluorophores.

Donor and acceptor fluorophore combinations can be selected from fluorophores with known FRET relationships. In certain instances, a donor fluorophore excitation maximum of about 300 nm to about 700 nm. Such wavelengths include near-ultraviolet (near-UV) to red wavelengths. In certain instances, donor fluorophores can have an excitation maximum in the range of near-UV (˜300 nm to ˜380 nm), violet (˜380 nm to ˜440 nm), blue (˜440 nm to ˜485 nm), cyan (˜485 nm to ˜510 nm), green (˜510 nm to ˜565 nm), yellow (˜565 nm to ˜ 590 nm), orange (˜590 nm to ˜625 nm), or red (˜625 nm to ˜700 nm). Generally, the emission maximum is at a higher wavelength than the excitation maximum, and resultant emission maxima of donor fluorophores in accordance with certain embodiments can be 380 to 440 nm, 440 to 485 nm, 485 nm to 510 nm, 510 to 565 nm, 565 to 590 nm, 590 to 625 nm, or 625 to 725 nm, depending on the excitation and Stokes Shift of a particular fluorophore. Exemplary chromophores include bodipy chromophores, aryl or heteroaryl chromophores, coumarin fluorophores, pyrene fluorophores, and triphenyl fluorophores. Such fluorophores are described in one or more of U.S. Pat. Nos. 11,702,547, 11,643,556, 11,485,825, 11,214,688, 11,209,438, 10,851,212, 10,844,228, 10,703,864, 10,663,476, 10,648,900, 10,240,004, 10,073,093, 10,018,640, 9,797,899, and 9,758,625; US Patent Publication Nos: 2023/0152327, 2022/0348770, 2021/0032474, and 2020/0225143, and U.S. application Ser. Nos. 18/201,940, 18/201,649, and 18/120,554; the disclosures of which are hereby incorporated by reference in their entireties.

As noted above, FRET utilizes an acceptor fluorophore with an excitation distribution that includes the emission maximum of the selected donor fluorophore. In certain embodiments, the emission maximum of the selected donor fluorophore is approximately equal to (e.g., +25 nm) the excitation maximum of the acceptor fluorophore. In certain embodiments, the one or more acceptor fluorophores has an excitation maximum ranging from 450 to 750 nm. Such maxima can include excitation maxima in the regions of blue (˜440 nm to ˜485 nm), cyan (˜485 nm to ˜510 nm), green (˜510 nm to ˜565 nm), yellow (˜565 nm to ˜ 590 nm), orange (˜590 nm to ˜625 nm), or red (˜625 nm to ˜740 nm), depending on a particular type of acceptor fluorophore, the desired emission maximum, and its Stokes Shift. Given the excitation and Stokes Shift, various acceptor fluorophores can have an emission maximum of approximately 500 nm to 875 nm, which generally fall into the cyan to near-infrared (near-IR) ranges of light, such as 475 nm to 600 nm, 600 to 700 nm, or 700 to 800 nm.

These acceptor fluorophores can be selected from rhodamine, a perylene, a diimide, a coumarin, a xanthene, a cyanine, a polymethine, a pyrene, a thiazine, an acridine, a dipyrromethene borondifluoride, a napthalimide, a phycobiliprotein, a peridinum chlorophyll protein, conjugates thereof, and combinations thereof. In certain embodiments, the acceptor fluorophore (A) is a cyanine dye, a xanthene dye, a coumarin dye, a thiazine dye or an acridine dye. In some instances, the acceptor fluorophore (A) is selected from DY 431, DY 485XL, DY 610, DY 640, DY 654, DY 682, DY 700, DY 701, DY 704, DY 730, DY 731, DY 732, DY 734, DY 752, DY 778, DY 782, DY 800, DY 831 and diethylamino coumarin. Fluorescent dyes of interest include, but are not limited to, Texas Red, Cascade Blue, Cascade Yellow, coumarin, Cy5, Cy5.5, Cy-Chrome, phycoerythrin, PerCP (peridinin chlorophyll-a Protein), PerCP-Cy5.5, Lucifer Yellow, Marina Blue, Oregon Green 488, Alexa Fluor 350, Alexa Fluor 430, Alexa Fluor 488, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY 576/589, BODIPY 581/591, BODIPY 630/650, BODIPY 650/665, BODIPY R⁶G, BODIPY TMR, BODIPY TR, conjugates thereof, and combinations thereof. Lanthanide chelates of interest include, but are not limited to, europium chelates, terbium chelates and samarium chelates. In some embodiments, the polymeric dye structure includes a multichromophore linked to an acceptor fluorophore selected from Cy5, Cy5.5, Cy7, Alexa488, Alexa 647 and Alexa700. In certain embodiments, the polymeric dye structure includes a multichromophore linked to an acceptor fluorophore selected from Dyomics dyes (such as DY 431, DY 610, DY 633, DY 640, DY 651, DY 654, DY 682, DY 700, DY 701, DY 704, DY 730, DY 731, DY 732, DY 734, DY 752, DY 754, DY 778, DY 782, DY 800 or DY 831), and diethylamino coumarin. In certain cases, the acceptor fluorophore (A) is selected from Texas Red, California Red, iFluor594, Cascade Blue, Cascade Yellow, coumarin, Cy5®, Cy5.50, Cy7®, Cy-Chrome, DyLight 350, DyLight 405, DyLight 488, DyLight 594, DyLight 633, DyLight 649, DyLight 680, DyLight 750, DyLight 800, phycoerythrin, PerCP (peridinin chlorophyll-a Protein), PerCP-Cy5.5, Lucifer Yellow, Marina Blue, Oregon Green 488, Alexa Fluor® 350, Alexa Fluor® 430, Alexa Fluor® 488, Alexa Fluor® 594, Alexa Fluor® 633, Alexa Fluor® 647, Alexa Fluor® 660, Alexa Fluor@ 680, 7-amino-4-methylcoumarin-3-acetic acid, BODIPY® 576/589, BODIPY® 581/591, BODIPY® 630/650, BODIPY® 650/665, BODIPY@ R⁶G, BODIPY® TMR, BODIPY® TR, conjugates thereof and combinations thereof. Various dyes are known in the art and/or described in one or more of U.S. Pat. Nos. 11,668,716, 11,624,089, 11,407,773, 11,371,078, 11,214,688, 11,130,865, 11,119,107, 10,605,813, 10,228,375, 10,053,484, 9,863,947, 9,797,899, 9,719,998, 9,551,029, 9,493,656, 9,383,353, 9,228,225, 9,034,920, 8,753,814, 8,163,910, 8,030,096, 7,767,829, 7,704,284, 7,563,907, 7,402,671, 7,256,291, 7,038,063, 6,750,346, 6,743,568, 6,399,392, 6,225,050, 6,191,278, 6,048,982, 5,286,803, and 4,945,171; and US Patent Publication Nos: 2020/0277670, 2011/010575, 2006/0199949, and 2003/0165942; the disclosures of which are hereby incorporated by reference in their entireties.

Where desired, the donor and/or acceptor fluorophores present in dye structures of embodiments of the invention may be substituted with one or more water solubilizing groups (WSG), e.g., as defined above.

Dye structures finding use in embodiments of the invention are further described in U.S. Pat. Nos. 11,702,547; 11,214,688; and 10,844,228; as well as published United States Patent Application Publication Nos. 2022/0348770 and 2023/0152327; and pending United States Patent Application Ser. Nos.: 18/120,554; 18/127,568; and Ser. No. 18/201,940; the disclosures of which are herein incorporated by reference.

### Quenchers

As described above a nucleic acid probe may include a quencher 106 in an energy transfer relationship with a one or more acceptor fluorophores 112. A quencher 106 can be linked to an end (e.g., 5′-end or 3′-end) of nucleic acid 102. In certain embodiments, the quencher 106 is linked to a 3′-end of nucleic acid 102. Certain embodiments include an internal quencher 106, wherein quencher 106 is linked to a nucleotide not located at an end. Internal quenchers can be located 7 to 15 nucleotides away from dye structure (e.g., 7 to 15 nucleotides from the 5′-end of the nucleic acid). In some specific embodiments, an internal quencher is located 9 or 10 nucleotides from a 5′-end of the nucleic acid. Internal quenchers can be used with a terminal quencher (e.g., a quencher at a 5′- or 3′-end) or instead of a terminal quencher.

Quenchers can include any convenient absorbative moiety. The quencher can be a small molecule quencher. In certain instances, the quencher is a fluorescent dye with an emission distribution significantly longer than the emission maximum of the donating fluorophore (e.g., an acceptor and/or donor fluorophore described above). In certain embodiments, the quencher is a non-fluorescent moiety. In certain embodiments, the absorption and/or excitation distribution of the quencher includes with the emission maximum of the acceptor fluorophore as described above (e.g., approximately 500 nm to 875 nm). The absorption and/or excitation distribution of the quencher can further include with the emission maximum of the donor fluorophore as described above (e.g., approximately 450 nm to 725 nm). As such, quenchers may absorb light from approximately 450 nm to approximately 875 nm, including the ranges of 440 to 485 nm, 485 nm to 510 nm, 510 to 565 nm, 565 to 590 nm, 590 to 625 nm, 625 to 740 nm, or 740 to 875 nm.

The quencher can be selected from a Black Hole Quencher (BHQ), a QSY quencher, an lowa Black quencher, or a QXL quencher. In certain embodiments, the quencher is selected from QSY-7, QSY-9, QSY-21, QSY-35, BHQ-1, BHQ-2, BHQ-3, lowa Black FQ, lowa Black RQ, QXL 490, QXL 570, QXL 670, 4-(dimethylamino)-azobenzene-4′-carboxylic acid (Dabcyl), tetramethylrhodamine (TAMRA), a phosphoramidite quencher, an azo quencher, an anthraquinone quencher, conjugates thereof, and combinations thereof.

The specific quencher used can be selected based on parameters of interest, including (but not limited to) the excitation maximum, the emission wavelength maximum, the Stokes shift, the extinction coefficient, and/or any other relevant factor.

Such quenchers are described in one or more of U.S. Pat. Nos. 11,242,554, 10,886,006, 9,540,515, 8,916,345, 8,410,255, 7,956,169, 7,879,986, 7,803,536, 7,439,341, 7,166,715, 7,019,129, 6,790,945, 6,699,975, 6,399,392, and 6,323,337; the disclosures of which are hereby incorporated by reference in their entireties.

### Additional Aspects of Probes

Certain embodiments of probes can include features to increase binding or hybridization efficacy with a target sequence. Such modifications can include a minor groove binder (MGB). Certain embodiments can include one or more, two or more, or any other number of MGBs on a probe. MGBs can be linked to a 5′-end or a 3′-end of a nucleic acid. For the purposes of the present disclosure, a molecule is considered an MGB if it is capable of binding within the minor groove of double stranded DNA with an association constant of 10-3 M-1 or greater. This type of binding can be detected by well-established spectrophotometric methods, such as ultraviolet and nuclear magnetic resonance (NMR) spectroscopy and also by gel electrophoresis.

MGBs can include certain naturally occurring compounds such as netropsin, distamycin, lexitropsin, mithramycin, chromomycin A3, olivomycin, anthramycin, sibiromycin, as well as further related antibiotics and synthetic derivatives. Certain bisquarternary ammonium heterocyclic compounds, diarylamidines such as pentamidine, stilbamidine, berenil, CC-1065 and related pyrroloindole and indole polypeptides, Hoechst 33258, 4′-6-diamidino-2-phenylindole (DAPI). Various oligopeptides consisting of naturally occurring or synthetic amino acids are minor groove binder compounds, including 1,2-dihydro-(3H)-pyrrolo[3,2-e] indole-7-carboxylate (CDPI) and N-methylpyrrole-4-carbox-2-amide (MPC). Certain embodiments include a dimer, a trimer, a tetramer and a pentamer of CDPI. Additional embodiments include a dimer, a trimer, a tetramer and a pentamer of MPC. Additional examples of MGBs can be found in U.S. Pat. Nos. 5,801,155 and 7,582,739; the disclosures of which are hereby incorporated by reference in their entireties.

Probes for nucleic acid detection can be assembled as a panel of probes for multiplex detection. In such instances, the nucleic acid 102 of each probe is complementary to a different target sequence—e.g., a first nucleic acid is complementary to a first target sequence, and a second nucleic acid is complementary to a second target sequence, where the first target sequence is different from the second target sequence. The first and second target sequences may be located on the same molecule (e.g., chromosome) or on different molecules. In certain instances, the first and second target sequences are located within the same amplicon (e.g., the region amplified by a set of primers). Additionally, to differentiate between probes within a panel, different combinations of donor and/or acceptor fluorophores can be used for each probe. Certain embodiments use the same donor fluorophores but use acceptor fluorophores with different emission wavelengths. Such situations allow probes to be detected simultaneously with only one excitation source. Other options for simultaneous detection can include donor fluorophores with similar excitation and emission distributions. Certain embodiments possess probes using donor and/or acceptor fluorophores with different excitation or emission distributions. In such situations, excitation or detection can occur asynchronously. One of skill in the art will understand how to select which donor and/or acceptor fluorophores and the appropriate excitation and detection.

A panel of probes can include as many probes as possible that can be discerned via simultaneous and/or asynchronous detection. For example, a finite number of fluorophores may have the same excitation maximum but different emission maxima, thus limiting the amount of simultaneous detection. Certain panels may thus use multiple sets of such fluorophores, such that a first excitation source will allow detection of several probes and a second excitation source will allow detection of several more probes. Examples of such fluorophores can be found in U.S. patent application Ser. Nos. 18/201,940 and 18/120,554; the disclosures of which are hereby incorporated by reference in their entireties.

The number of distinct probes in a given panel may vary. In some instances, the number is 2 or more, such as 5 or more, including 10 or more, e.g., 25 or more, 50 or more, 75 or more, etc., where in some instances the number may be 1000 or less, such as 500 or less, including 250 or less, e.g., 200 or less, 150 or less, including 100 or less. The number of probes in a panel may be 5-10 probes, 5-15 probes, 5-25 probes, 10-20 probes, 10-25 probes, 10-30 probes, 10-35 probes, 10-40 probes, 10-45 probes, 10-50 probes, 15-25 probes, 15-35 probes, 15-45 probes, and/or any other range of probes. A panel of probes may be targeted to a particular set of genes, such that the panel has a diagnostic or prognostic ability for a disease, such as a cancer. Additional panels can focus on the Recommended Uniform Screening Panel (RUSP). RUSP is a list of disorders that the Secretary of the Department of Health and Human Services recommends as part of universal newborn screening programs. Certain panels can target microbial traces in a sample, such as to diagnose an infectious agent and optionally its variants. For example, a panel may be used to identify tuberculosis and some of its antibiotic resistant variants.

A panel of probes can be added to a single reaction, such as a real time or quantitative PCR reaction. In certain instances, a panel is provided as a collection of individual probes, where each probe is packaged or portioned individually, while in other instances, a panel is included as a pooled collection of all probes in the panel. In either pooled or individually packaged instances, a panel may include components to assist with stability and/or detection. Such components can include one or more of a buffer, a primer, a polymerase, nucleotides, a thickening agent, and a fluorescence stabilizer to avoid photobleaching. A panel, with or without additional components can be provided dry (e.g., lyophilized) or in a suitable medium. Suitable media can be aqueous (e.g., water-based) or organic. In a pooled embodiment, the probes may be optimized or tuned.

As discussed above, the tunability of embodiments can assist in multiplex applications. Such tunability can compensate for issues in detection—e.g., reduced emission and/or signal brightness from certain fluorophores- and to compensate for variation within a sample—e.g., simultaneous detection of small and large quantities of targets within a sample. As such, probes within a panel of probes may have dye structures with varying numbers of donor and/or acceptor fluorophores to compensate for variation in brightness and/or emissions from probes within a panel.

Dye structures of certain embodiments may vary. In some instances, dye structures of certain embodiments include: a polymeric backbone; one or more pendant donor fluorophores each independently linked to a repeat unit of the polymeric backbone, and one or more pendant acceptor fluorophores linked to a repeat unit of the polymeric backbone, where pendant donor and acceptor fluorophores are in energy transfer relationship. As such, dye structures of some embodiments include one or more pendant donor fluorophores and one or more pendant acceptor fluorophores configured in energy-receiving proximity to the one or more pendant donor fluorophores, e.g., where both are linked to a common polymeric backbone. In some embodiments, a plurality of pendant donor fluorophores are present and are configured in energy-transferring proximity to a pendant acceptor fluorophore(s), where in some instances the plurality of pendant donor fluorophores ranges from 2 to 20, such as 2 to 15, e.g., 2 to 10. The term “pendant group” refers to a sidechain group that is connected to the backbone but which is not part of the backbone itself. In embodiments of the dye structures, the donor fluorophore is capable of transferring energy to a linked acceptor fluorophore. As such, the subject dye structures include a linked acceptor signaling fluorophore in energy-receiving proximity to the donor fluorophore system, i.e., in energy-receiving proximity to at least one linked donor fluorophore. A particular configuration of pendant groups can be determined and controlled by the arrangement of the repeat units of the underlying polymeric backbone (also referred to herein as “modular scaffold” to which the pendant groups are attached. The dye structures can include a plurality of water solubilizing groups attached to the scaffold and/or the pendant groups at any convenient locations to provide a water soluble polymeric dye. The polymeric backbone, i.e., modular scaffold, can be composed of repeat units which form a polymeric backbone having sidechain groups to which the pendant groups can be attached. The repeat units can be arranged in a variety of configurations to provide for a dye structure having desirable spectroscopic properties. The distances and arrangement between sites for covalent attachment of the pendant donor fluorophores and the acceptor fluorophore(s) (when present) can be controlled to provide for desirable energy transfer processes.

As mentioned above, where desired, the polymeric backbone and/or pendant fluorophores (i.e., donor and acceptor fluorophores) may include one or more water solubilizing groups (WSG). In some cases, the WSGs are pendant groups connected directly to the polymeric backbone, e.g., as sidechains of a polymeric backbone. In certain cases, the WSGs are substituent groups attached to a pendant donor fluorophore or pendant acceptor fluorophore. In some instances, each of the pendant donor fluorophore groups is substituted with one or more WSG. As used herein, the terms “water solubilizing group”, “water soluble group” and WSG are used interchangeably and refer to a group or substituent that is well solvated in aqueous environments e.g., under physiological conditions, and which imparts improved water solubility upon the molecule to which it is attached. A WSG can increase the solubility of a given dye structures in a predominantly aqueous solution, as compared to a control dye which lacks the WSG. The water solubilizing groups may be any convenient hydrophilic group that is well solvated in aqueous environments. A water soluble dye structure of the present disclosure has solubility under aqueous conditions that makes it especially suitable for application to a variety of biological assays. A variety of water soluble polymer groups can be adapted for use in the WSG of the subject dyes. Any convenient water solubilizing groups (WSG's) may be included in the dyes described herein to provide for increased water-solubility, e.g., as described above.

As summarized above, dye structures of embodiments of the invention include a plurality of pendant donor fluorophores and one or more pendant acceptor fluorophores. In some instances, the number of donor fluorophores exceeds the number of acceptor fluorophores. In certain embodiments of the subject dye structures, the ratio of donor fluorophores to acceptor fluorophores is selected from 5:1, 6:1, 7:1, 8:1, 9:1, 10:1, and 20:2. In certain cases, the ratio of donor fluorophores to acceptor fluorophores is 5:1. In certain cases, the ratio of donor fluorophores to acceptor fluorophores is 6:1. In certain cases, the ratio of donor fluorophores to acceptor fluorophores is 7:1. In certain cases, the ratio of donor fluorophores to acceptor fluorophores is 8:1. In certain cases, the ratio of donor fluorophores to acceptor fluorophores is 9:1. In certain cases, the ratio of donor fluorophores to acceptor fluorophores is 10:1.

As mentioned above, in dye structures of the invention, pendant donor and acceptor fluorophores are in energy transfer relationship. As such, in embodiments of the invention, excitation of the donor can lead to energy transfer to, and emission from, the covalently attached acceptor signaling fluorophore. Mechanisms for energy transfer between the donor chromophores to a linked acceptor signaling fluorophore include, for example, resonant energy transfer (e.g., Förster (or fluorescence) resonance energy transfer, FRET), quantum charge exchange (Dexter energy transfer) and the like. These energy transfer mechanisms can be relatively short range; that is, close proximity of chromophores of the light harvesting multichromophore system to each other and/or to an acceptor fluorophore provides for efficient energy transfer. Under conditions for efficient energy transfer, amplification of the emission from the acceptor fluorophore can occur where the emission from the luminescent acceptor fluorophore is more intense when the incident light (the “pump light”) is at a wavelength which is absorbed by, and transferred from, the chromophores of the light harvesting chromophore than when the luminescent acceptor fluorophore is directly excited by the pump light. By “efficient” energy transfer is meant 10% or more, such as 20% or more or 30% or more, 40% or more, 50% or more, of the energy harvested by the donor chromophores is transferred to the acceptor. By “amplification” is meant that the signal from the acceptor fluorophore is 1.5× or greater when excited by energy transfer from the donor light harvesting chromophore system as compared to direct excitation of the acceptor fluorophore with incident light of an equivalent intensity. The signal may be measured using any convenient method. In some cases, the 1.5× or greater signal refers to an intensity of emitted light. In certain cases, the 1.5× or greater signal refers to an increased signal to noise ratio. In certain embodiments of the dye structure, the acceptor fluorophore emission is 1.5 fold greater or more when excited by the chromophore as compared to direct excitation of the acceptor fluorophore with incident light, such as 2-fold or greater, 3-fold or greater, 4-fold or greater, 5-fold or greater, 6-fold or greater, 8-fold or greater, 10-fold or greater, 20-fold or greater, 50-fold or greater, 100-fold or greater, or even greater as compared to direct excitation of the acceptor fluorophore with incident light.

The dye structures of embodiments of the invention may be of any convenient molecular weight (MW). In some cases, the MW of the dye structure may be expressed as an average molecular weight. In some instances, the dye structure has an average molecular weight in the range of 500 to 500,000, such as from 1,000 to 100,000, from 2,000 to 100,000, from 10,000 to 100,000 or even an average molecular weight in the range of 50,000 to 100,000 daltons. In some instances, the polymeric fluorescent dyes have a molecular weight ranging from 5 to 75 kDa, 10 to 50 kDa, such as 15 to 45 kDa.

In embodiments, the subject dye structures provide for fluorescence emissions from acceptor fluorophores that are brighter than the emissions which are possible from such fluorescent dyes in isolation. The emission of the dye structure can have a brightness of 50 mM-1 cm-1 or more, such as 60 mM 1 cm-1 or more, 70 mM-1 cm-1 or more, 80 mM-1 cm-1 or more, 90 mM-1 cm-1 or more, 100 mM-1 cm-1 or more, 150 mM-1 cm-1 or more, 200 mM-1 cm-1 or more, 250 mM-1 cm-1 or more, 300 mM-1 cm-1 or more, or even more. In certain instances, the emission of the dye structure has a brightness that is at least 5-fold greater than the brightness of a directly excited acceptor fluorophore, such as at least 10-fold greater, at least 20-fold greater, at least 30-fold greater, at least 50-fold greater, at least 100-fold greater, at least 300-fold greater, or even greater than the brightness of a directly excited acceptor fluorophore.

In addition to attributes such as described above, dye structures of embodiments of the invention may have one or more additional desirable spectroscopic properties, such as a particular emission maximum wavelength, extinction coefficient, quantum yield, and the like.

A variety of emission profiles depend on a variety of factors such as the selected co-monomers, linking groups, substituents and linked acceptor fluorophores of which the dye structures are composed.

In some embodiments, the dye structure can include segments having formulae as described in one or more of PCT/US2019/024662 (published as WO 2019/191482) and U.S. patent application Ser. Nos. 18/201,940 and 18/120,554; the disclosures of which are herby incorporated by reference in their entireties. Further details regarding WSG groups that may be found in dye structures of embodiments of the invention are found in PCT application serial no. PCT/US2019/024662 published as WO 2019/191482.

A resulting and general structure of nucleic acid probes is illustrated in the following formula:

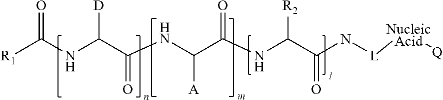

wherein:
each D is independently a pendant donor fluorophore (e.g., as described herein);
each A is independently an acceptor chromophore (e.g., as described herein);
I, m, and n each independently an integer from 1 to 20;
L is a linker;
R₁ and R₂ are independently selected from a terminal group, a polymer segment, a donor chromophore group, a water solubilizing group, a linker, and a linker to a specific binding member;
Nucleic Acid is a nucleic acid having a sequence complementary to a target nucleic acid (e.g., as described herein); and
Q is a quencher (e.g., as described herein).

In some cases of the foregoing formula, I, m, and n are each independently 1 to 10 such as 2 to 20, 3 to 10 or 3 to 6. In some instances of the foregoing formula, n is an integer from 2 to 20, such as 3 to 20, 4 to 20, 5 to 20, 5 to 15 or 5 to 12. In certain embodiments of the foregoing formula, m is 1.

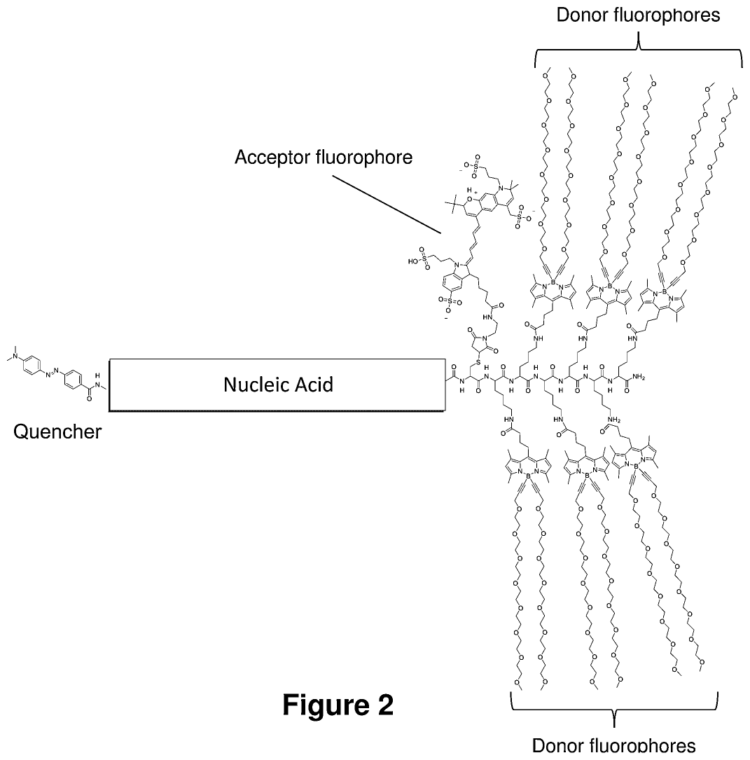

One exemplary (and non-limiting) representation of a nucleic acid probe in accordance with certain embodiments is illustrated in FIG. 2. The example according to FIG. 2 provides a nucleic acid with a dye structure attached to a first end of the nucleic acid and a quencher attached to a second end of the nucleic acid. The dye structure comprises a non-conjugated polymeric backbone formed of amino acids—e.g., peptide structure. This example provides for six donor fluorophores and one acceptor fluorophore, in accordance with the foregoing description.

### Methods

Aspects of the invention include methods of evaluating a sample for the presence of a target nucleic acid. Aspects of the methods include producing a reaction mixture that includes a sample and a nucleic acid probe. Such probes have been described in the foregoing disclosure and may be used in producing the reaction mixture. In certain instances, the sample includes, is suspected of including, may include, or does not include a nucleic acid. In certain situations, the sample includes one or more copies of a target nucleic acid to which the nucleic acid probe is complementary. In certain embodiments, the nucleic acid probe hybridizes to any copies of the target nucleic acid in the sample. Additional embodiments monitor the reaction mixture for a signal emitted from the dye structure associated with the nucleic acid probe to assay the sample for the presence of the target nucleic acid.

In some instances, the method can include illuminating the reaction mixture with an excitation wavelength of the one or more donor fluorophores. The specific wavelength, bandwidth, intensity, or other parameter may be selected by one in the art and dependent on which donor fluorophore is part of the nucleic acid probe.

In some instances, the reaction mixture further includes a polymerase with 5′-3′ nuclease activity and the monitoring of the reaction mixture includes performing a nucleic acid amplification reaction. Nucleic acid amplification can include isothermal amplification and polymerase chain reaction (PCR). In an amplification reaction as described herein, the polymerase with 5′-3′ nuclease activity polymerizes a new strand based on a target sequence or target region. When the polymerase encounters a hybridized nucleic acid probe, the nuclease activity hydrolyzes the nucleic acid that is part of the nucleic acid probe. This hydrolysis liberates the dye structure from the quencher and allows a signal to be emitted from the dye structure.

As will be known to one of skill in the art, PCR includes providing a pair of amplification primers and deoxynucleotide triphosphates to the reaction mixture, where the first amplification primer contains a sequence complementary to a region of one strand of the target nucleic acid and is capable of priming the synthesis of the complementary strand and the second primer contains a sequence complementary to a region of the second strand of the target nucleic acid and is capable of priming the synthesis of a strand which is complementary thereto. In such embodiments, the polymerase exhibiting 5′-3′ nuclease activity amplifies the target nucleic acid between the amplification primers is amplified, and the nucleic acid probe is complementary to a region of the target nucleic acid located between the pair of amplification primers. PCR further can further include cycling the reaction temperature of the sample to a first temperature suitable to denature the target nucleic acid, then to a second temperature suitable to allow hybridization of the pair of amplification primers and the nucleic acid probe to the target nucleic acid, and then to a third temperature suitable for the polymerase to amplify the target nucleic acid. The cycling process may be iterated for any number of iterations sufficient to allow a signal to be emitted from a dye structure. Certain embodiments iterate the cycling of the reaction temperature for 4-35 iterations, including at least 4 iterations, at least 5 iterations, at least 10 iterations, at least 15 iterations, at least 20 iterations, or at least 25 iterations.

Reaction temperatures for PCR may be selected as appropriate for a particular reaction type, reaction mix, and/or components included within the reaction mix. In PCR, the first temperature to denature can be at or above a temperature to separate any double stranded nucleic acids. This first temperature can be at or near 100° C., such as 90° C., 92° C., 94° C., 95° C., 96° C., 97° C., 98° C., 99° C., or 100° C. However lower temperatures may be used in certain circumstances that can be appreciated by one of skill in the art. A second temperature that is suitable to allow hybridization or annealing of primers is typically a temperature at or below the melting temperature (Tm) of a primer and/or probe. Typically, this range is between 55° C. and 72° C., but certain embodiments may use a lower temperature. Additionally, this second temperature can be adjusted between iterations, such as in step-up PCR (increasing temperature with each iteration), touchdown PCR (decreasing temperature with each iteration), and/or any other cycling strategy for improving sensitivity and/or detection. Finally, the third temperature suitable for extension is dependent on the particular enzyme to ensure processivity and/or efficiency in extension or polymerization of a new nucleic acid strand. This temperature is typically approximately 72° C., but it can be altered depending on the enzyme. As such, the third temperature can be selected from approximately 65° C. to approximately 80° C., including 70° C., 72° C., 75° C., and/or 78° C.

Certain instances implement probes, as described herein, in real time PCR, including quantitative PCR (qPCR). While real time and qPCR can use a non-specific dye to quantify amplification, these methodologies can measure off-target amplification, thus have limited accuracy. Probes, as described herein, improve qPCR accuracy by detecting specific amplicons. Such probes hybridize or anneal to a target sequence (i.e., a probe's complementary sequence) during the same phase as primer hybridization. During an extension phase, a polymerase with 5′-3′ exonuclease activity can hydrolyze or digest the nucleic acid portion of a probe. The hydrolysis liberates the scaffolded chromophore or dye structure from the probe and allows the dye structure to diffuse within a reaction mixture. The liberation breaks an energy transfer relationship between acceptor fluorophores on the dye structure and the quencher, thus allowing a fluorescent signal to be measured or imaged. In qPCR reactions, imaging or detection can occur continuously throughout the reaction or at a singular, time during the temperature cycling. For example, imaging can occur after extension but before denaturation. However, the imaging or detection can occur during denaturation and/or during hybridization. Details regarding real time PCR and/or qPCR finding use in embodiments of the invention are further described in U.S. Pat. Nos. 11,001,877, 9,944,978, 8,697,362, 8,137,616, 8,017,380, and 7,101,663; as well as published United States Patent Application Publication Nos.: 2020/0370097, 2012/0088691, 2011/0300544, 2005/0053950; the disclosures of which are herein incorporated by reference in their entireties.

Isothermal amplification can proceed via various methods known in the art, including Nucleic Acid Sequence-based Amplification (NASBA), Loop-mediated Isothermal Amplification (LAMP), Strand Displacement Amplification (SDA), Recombinase Polymerase Amplification (RPA) and Rolling Circle Amplification (RCA). In addition to a polymerase with 5′-3′ nuclease activity, such reaction mixtures may include one or more primers, a ligase, a recombinase, deoxyribonucleotides, ribonucleotides, one or more adapters, and various other components that are known to a person in the art. Isothermal amplification reaction temperatures may depend on type of reaction, enzyme, and/or any other factor. In certain embodiments, the reaction occurs between approximately 30° C. and 45° C., such as 32° C., 35° C., 37° C., 40° C., and 42° C. Certain embodiments use 37° C. as the reaction temperature for an isothermal amplification reaction.

Nucleic acid amplifications, such as those described herein may further include components to assist the reaction, including a buffer, a solvent, and/or any other component. Such buffers can include HEPES, PBS, SSC, Tris, any other buffer for a particular enzyme, and combinations thereof. Solvents can also be used to increase specificity, reaction efficiency, and/or any other property to assist with such reactions. A common solvent used in many reactions is dimethylsulfoxide (DMSO). Additional components may be part of a reaction mixture, as one of skill in the art would understand.

Additionally, methods described herein can be used in multiplexed reactions with a second nucleic acid probe as described herein. With a second probe, the associated nucleic acid is complementary to a second target sequence, which is different from the first target sequence. When used in an amplification reaction, the second probe can hybridize between the pair of amplification primers of the first nucleic acid probe or to a region between a second pair of amplification primers. In certain multiplex embodiments, the number of different target nucleic acids that are simultaneously assayed by vary, where in some instances the number of different target nucleic acids that are simultaneously assayed 2 or more, such as 5 or more, including 10 or more, e.g., 25 or more, 50 or more, 75 or more, etc., where in some instances the number may be 1000 or less, such as 500 or less, including 250 or less, e.g., 200 or less, 150 or less, including 100 or less. The number of target nucleic acids to be assayed may be 5-10 targets, 5-15 targets, 5-25 targets, 10-20 targets, 10-25 targets, 10-30 targets, 10-35 targets, 10-40 targets, 10-45 targets, 10-50 targets, 15-25 targets, 15-35 targets, 15-45 targets, and/or any other range of targets.

In some embodiments, the sample is anything that does or may contain nucleic acids. Such samples can be biological or environmental. Biological samples include animals, plants, fungi, bacteria, viruses, and/or archaea. Samples can be a whole organism, a single cell, or another portion thereof. Certain samples can be from one tissue, one organ, a biological product (e.g., blood), or any combination thereof. In certain embodiments, the biological sample is collected as saliva, feces, urine, sweat, sebum, mucus, semen, milk, blood, and/or any other biological product. Blood samples can be collected as peripheral whole blood, peripheral whole blood in which erythrocytes have been lysed prior to cell isolation, cord blood, bone marrow, density gradient-purified peripheral blood mononuclear cells. Certain biological samples can include cell and/or tissue culture, such as CD34+ cells, HEK cells, Hela cells, and/or any other cell line. Tissue samples can include normal tissue (e.g., liver, kidney, brain, skin, mammary, adipose, bone, bone marrow, etc.) or abnormal tissue (e.g., neoplastic, cancer, tumor, diseased, etc.)

Environmental samples can be used to identify the presence of certain species or organisms without direct sightings or other evidence of existence. Such samples can include samples collected from air, water, sewer, ground, toilets, sinks, doorknobs, and/or any other non-biological location or material.

Fluorescence in a sample can be measured using a fluorimeter. In some cases, excitation radiation, from an excitation source having a first wavelength, passes through excitation optics. The excitation optics cause the excitation radiation to excite fluorophores within the reaction mixture. In response, fluorescently labelled targets in the sample emit radiation which has a wavelength that is different from the excitation wavelength. Collection optics then collect the emission from the reaction mixture. The device can include a temperature controller to maintain the sample at a specific temperature while it is being scanned. In certain instances, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In some embodiments, the method of evaluating a sample for the presence of a target nucleic acid further includes detecting fluorescence in a flow cytometer. In some embodiments, the method of evaluating a sample for the presence of a target nucleic acid further includes imaging the labelling composition contacted sample using fluorescence microscopy. Fluorescence microscopy imaging can be used to identify a polymeric dye conjugate-target nucleic acid binding complex in the contacted sample to evaluate whether the target analyte is present. Microscopy methods of interest that find use in the subject methods include laser scanning confocal microscopy.

As certain methods for detecting a target nucleic acid utilize nucleic acid amplification, fluorescence can be detected via a real time thermal cycler or a thermal cycler with an attached excitation and detection array to monitor fluorescence emission from the reaction mixture.

### Hybridization Products

Certain embodiments of this disclosure include products generated using nucleic acid probes as disclosed herein. Such products can include a probe hybridized to a target nucleic acid. Such products can include a first nucleic acid and a second nucleic acid, each comprising a plurality of nucleotides forming a chain from a 5′-end to a 3′-end. Additionally, the first nucleic acid is complementary to at least a portion of the second nucleic acid. The first nucleic acid can be a nucleic acid probe as described herein and includes a dye structure and quencher as described herein. The first nucleic acid can include a second quencher, minor groove binder, water solubilizing group, etc. as described above.

The second nucleic acid can include a target nucleic acid. Such a nucleic acid can be DNA, RNA, or another form of nucleic acid (e.g., as described herein). Such targets can be nuclear and/or genomic DNA, messenger RNA (mRNA), pre-mRNA, ribosomal RNA, transfer RNA, an amplicon, and/or any other nucleic acid that can be targeted and/or be of interest.

### Systems

Aspects of the invention further include systems for use in practicing the subject methods and compositions. A sample analysis system can include a chamber holding a sample as described herein. Systems of such embodiments can include an illumination source directed to impinge on the sample and a detector directed such that light emissions from the sample impinge on the detector. The sample can include a nucleic acid probe as described and includes or is suspected of including a target nucleic acid. Further embodiments of the system can include a heating element to alter temperature of the sample. Such heating elements can include a water bath or heating block and may use the thermoelectric effect to alter the temperature of the sample.

Further embodiments include a user interface to allow selection of a temperature, an illumination wavelength, a detection wavelength, and temperature cycling program. Such a user interface may be analog, digital, and/or graphical. Examples of such systems can include thermal cyclers, plate readers, flow cytometers, fluorescence microscope, and/or any other system that fulfills the description herein.

In certain aspects, the system may further include computer-based systems configured to detect the presence of the fluorescent signal. A “computer-based system” refers to the hardware means, software means, and data storage means used to analyze the information of the present invention. The minimum hardware of the computer-based systems of the present invention includes a central processing unit (CPU), input means, output means, and data storage means. A skilled artisan can readily appreciate that any one of the currently available computer-based systems are suitable for use in the subject systems. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

Other systems may find use in practicing the subject methods. In certain aspects, the system may be a fluorimeter or microscope loaded with a sample having a fluorescent composition of any of the embodiments discussed herein. The fluorimeter or microscope may include a light source configured to direct light to the assay region of the flow channel or sample field of view. The fluorimeter or microscope may also include a detector configured to receive a signal from an assay region of the flow channel or field of view, wherein the signal is provided by the fluorescent composition.

### Kits

Aspects of the invention further include kits for use in practicing the subject methods and compositions. The compositions, e.g., one or more probes (such as described above) of the invention can be included as reagents in kits either as starting materials or provided for use in, for example, the methodologies described above. A kit can include a [probe, e.g., as described above, and a container. Any convenient containers can be utilized, such as tubes, bottles, or wells in a multi-well strip or plate, a box, a bag, an insulated container, and the like. The subject kits can further include one or more components selected from a probe for a given target sequence, a support bound probe, a cell, a support, a biocompatible aqueous elution buffer, a control (positive and/or negative), etc., and instructions for use, as desired. A given kit may include reagents suitable for detection of a single target nucleic acid, or multiple reagents suitable for detection of two or more different target nucleic acid, e.g., where a given kit is configured for multiplex detection applications.

In certain embodiments, the kit finds use in evaluating a sample for the location of a target sequence, such as an in situ hybridization. As such, in some instances, the kit includes one or more components suitable for permeabilizing or lysing cells. The one or more additional components of the kit may be provided in separate containers (e.g., separate tubes, bottles, or wells in a multi-well strip or plate).

In certain aspects, the kit further includes reagents for performing nucleic acid amplification. Reagents of interest include, but are not limited to, buffers for reconstitution and dilution, buffers for contacting a sample with a nucleic acid probe, wash buffers, control reagents, enzymes (e.g., polymerases, polymerases with 5′-3′ nuclease activity, recombinases, ligases, etc.), nuclease-free water, nucleotide triphosphates, deoxynucleotide triphosphates, one or more primers, adapters, and combinations thereof. Further, the kit may include a cell permeabilizing reagent, such as methanol, acetone or a detergent (e.g., triton, NP-40, saponin, tween 20, digitonin, leucoperm, or any combinations or buffers thereof.

The compositions of the kit may be provided in a liquid composition, such as any suitable buffer. Alternatively, the compositions of the kit may be provided in a dry composition (e.g., may be lyophilized), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Utility

The scaffolded chromophores for nucleic acid detection and nucleic acid probes comprising the same, compositions, methods and systems as described herein may find use in a variety of applications, including diagnostic and research applications, in which the labelling, detection and/or analysis of a target of interest is desirable. Such applications include methodologies such as cytometry, microscopy, immunoassays (e.g. competitive or non-competitive), assessment of a free analyte, assessment of receptor bound ligand, and so forth. The compositions, system and methods described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the compositions and methods find use in applications where the evaluation of a sample for the presence of a target analyte is of interest.

In some cases, the methods and compositions find use in any assay format where the detection and/or analysis of a target from a sample is of interest, including but not limited to, quantitative PCR, flow cytometry, fluorescence microscopy, in situ hybridization, enzyme-linked immunosorbent assays (ELISAs), western blot analysis, magnetic cell separation assays and fluorochrome purification chromatography. In certain instances, the methods and compositions find use in any application where the fluorescent labelling of a target molecule is of interest. The subject compositions may be adapted for use in any convenient applications where nucleic acid probes find use.

The following example is offered by way of illustration and not by way of limitation.

### EXAMPLES

### Example 1—Real-Time PCR

Various nucleic acid probes will be incorporated into real-time PCR (rtPCR). rtPCR can be quantitative and referred to as quantitative PCR (qPCR). In rtPCR, a reaction mixture will be produced in accordance with a standard PCR reaction with the addition of a probe that can identify a specific sequence that can be present within the sample. A reaction mixture will include sufficient amounts of buffer, nucleotides (e.g., deoxyribonucleotides and/or ribonucleotides), a polymerase (e.g., taq polymerase), primers, template, and probe, such as the probe illustrated in FIG. 2. The reaction mixture may include the following components and amounts:

| | | |
|---|---|---|
| Template or sample | 10 | μL |
| Forward primer (5 μM) | 0.5 | μL |
| Reverse primer (5 μM) | 0.5 | μL |
| dNTPs (10 mM) | 0.5 | μL |
| 10X Buffer | 2.5 | μL |
| Taq polymerase (exo+) | 0.5 | μL |
| Probe (FIG. 2) (2.5 μM) | 0.5 | μL |
| Water | 10 | μL |
| Total | 25 | μL |

Once a reaction mixture is produced, the reaction mixture will be incubated in an appropriate thermal cycler-real-time thermal cyclers typically possess a light source and detector to allow excitation of a fluorophore and detection of any fluorophore emission. The thermal cycler will be cycled through temperatures to allow denaturation, primer and probe annealing, and primer extension. The cycling reaction may follow the following program:

| | |
|---|---|
| (1) Initial denaturation | 95° C. for 3 minutes |
| (2) Denaturation | 95° C. for 1 minute |
| (3) Annealing | 55° C. for 2 minutes |
| (4) Extension | 72° C. for 3 minutes |
| (5) Imaging/Detection | |
| (6) Cycle steps 2-5 for 10-45 cycles | |

During extension, the exonuclease activity of the polymerase will hydrolyze the nucleic acid, thus liberating the donor and acceptor fluorophores from the nucleic acid. Liberating the fluorophores will allow the fluorophores to diffuse away from the quencher. This diffusion will eliminate the energy-transfer relationship between the fluorophores and the quencher allowing for emission.

The reaction will be monitored with the appropriate excitation and emission maxima. Using the probe illustrated in FIG. 3, the appropriate excitation wavelength will include the excitation maximum of the six donor fluorophores. The detection wavelength will include the emission maximum of the acceptor fluorophore. The monitoring can occur consistently through the reaction program or occur during a separate detection or imaging step.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1-117. (canceled)

118. A method of assaying a sample for the presence of a target nucleic acid, the method comprising:
(a) producing a reaction mixture comprising:
the sample; and
a nucleic acid probe comprising:
a nucleic acid complementary to the target nucleic acid, wherein the nucleic acid comprises a plurality of nucleotides forming a chain from a 5′-end to a 3′-end,
a dye structure linked to a first end of the nucleic acid, wherein the dye structure comprises:
a non-conjugated polymeric backbone,
one or more donor fluorophores linked to the non-conjugated polymeric backbone, and
one or more acceptor fluorophores linked to the non-conjugated polymeric backbone, wherein the donor and acceptor fluorophores are in energy transfer relationship, and
a quencher linked to a second end of the nucleic acid, wherein the quencher and acceptor fluorophores are in energy transfer relationship; and
wherein the nucleic acid probe hybridizes to any copies of the target nucleic acid in the sample; and
(b) monitoring the reaction mixture for a signal emitted from the dye structure to assay the sample for the presence of the target nucleic acid.

119. The method of claim 118, further comprising illuminating the reaction mixture with an excitation wavelength of the one or more donor fluorophores.

120. The method of claim 118, wherein the reaction mixture further comprises a polymerase with 5′-3′ nuclease activity and wherein monitoring the sample further comprises performing a polymerase chain reaction (PCR) on the reaction mixture.

121. The method of claim 120, wherein the PCR comprises:
providing a pair of amplification primers and deoxynucleotide triphosphates to the reaction mixture;
wherein the first amplification primer contains a sequence complementary to a region of one strand of the target nucleic acid and is capable of priming the synthesis of the complementary strand,
wherein the second primer contains a sequence complementary to a region of the second strand of the target nucleic acid and is capable of priming the synthesis of a strand which is complementary thereto,
wherein the polymerase exhibiting 5′-3′nuclease activity amplifies the target nucleic acid between the amplification primers is amplified, and
wherein the nucleic acid probe is complementary to a region of the target nucleic acid located between the pair of amplification primers; and
cycling the reaction temperature of the sample to a first temperature suitable to denature the target nucleic acid, then to a second temperature suitable to allow hybridization of the pair of amplification primers and the nucleic acid probe to the target nucleic acid, and then to a third temperature suitable for the polymerase to amplify the target nucleic acid.

122. The method of claim 121, wherein monitoring the sample further comprises iterating the cycling the reaction temperature for at least 4 iterations.

123. The method of claim 118, wherein the reaction mixture further comprises a polymerase with 5′-3′ nuclease activity and wherein monitoring the sample further comprises performing an isothermal amplification reaction.

124. The method of claim 123, wherein the isothermal amplification reaction comprises utilizing one or more of the following: Nucleic Acid Sequence-based Amplification (NASBA), Loop-mediated Isothermal Amplification (LAMP), Strand Displacement Amplification (SDA), Recombinase Polymerase Amplification (RPA) and Rolling Circle Amplification (RCA).

125. The method of claim 118, wherein:
the reaction mixture further comprises a second nucleic acid probe,
wherein the second nucleic acid probe comprises:
a second nucleic acid complementary to a second target nucleic acid, wherein the second nucleic acid comprises a plurality of nucleotides forming a chain from a 5′-end to a 3′-end,
a second dye structure linked to a first end of the second nucleic acid, wherein the second dye structure comprises:
a non-conjugated polymeric backbone,
one or more donor fluorophores linked to the non-conjugated polymeric backbone, and
one or more acceptor fluorophores linked to the non-conjugated polymeric backbone, wherein the donor and acceptor fluorophores are in energy transfer relationship, and
a quencher linked to a second end of the nucleic acid, wherein the quencher and acceptor fluorophores are in energy transfer relationship; and
wherein the sample includes or is suspected of including a second target nucleic acid;
wherein the target sequence is different from the second target sequence; and
wherein the dye structure has a different emission maximum than the second dye structure.

126-133. (canceled)

134. The method of claim 118, wherein the one or more excitable donor fluorophores associated with the first nucleic acid probe and the one or more excitable donor fluorophores associated with the second nucleic acid probe have different excitation maxima.

135-142. (canceled)

143. The method of claim 118, wherein the one or more acceptor fluorophores associated with the first nucleic acid probe and the one or more acceptor fluorophores associated with the second nucleic acid probe have different emission maxima.

144-145. (canceled)

146. The method of claim 118, wherein the number of donor fluorophores exceeds the number of acceptor fluorophores in each of the first and second nucleic acid probes.

147-148. (canceled)

149. The method of claim 118, wherein the non-conjugated polymeric backbones comprise at least one of a peptide, a carbohydrate, a lipid, a peptoid, and a polynucleotide.

150. The method according to claim 149, wherein the non-conjugated polymeric backbones each comprise a peptide.

151. (canceled)

152. The method of claim 118, wherein the quenchers absorb light at the emission maximum of the one or more acceptor fluorophores.

153-156. (canceled)

157. The method of claim 118, wherein the first and second dye structures are linked to the 5′-ends of the first and second nucleic acids and the quenchers are linked to the 3′-ends of the first and nucleic acids.

158. The method of claim 118, wherein the first and second dye structures are linked to the 3′-ends of the first and second nucleic acids and the quenchers are linked to the 5′-ends of the first and second nucleic acids.

159. The method according to claim 157, wherein one or both of the first and second nucleic acid probes further comprises a second quencher linked to a nucleotide located 7 to 15 nucleotides away from the 5′-end of the nucleic acid.

160. The method according to claim 157, wherein the second quencher is linked to a nucleotide located 9 or 10 nucleotides away from the 5′-end of the nucleic acid.

161. The method according to claim 159, wherein the second quencher is selected from QSY-7, QSY-9, QSY-21, QSY-35, BHQ-1, BHQ-2, BHQ-3, Iowa Black FQ, lowa Black RQ, QXL 490, QXL 570, QXL 670, 4-(dimethylamino)-azobenzene-4′-carboxylic acid (Dabcyl), tetramethylrhodamine (TAMRA), a phosphoramidite quencher, an azo quencher, or an anthraquinone quencher.

162. The method of claim 118, wherein the reaction mix further comprises one or more of the following: a buffer, water, deoxynucleotide triphosphates, nucleotide triphosphates, a polymerase, a 5′-3′ nuclease, and a polymerase with 5′-3′ nuclease activity.

163-240. (canceled)
